(19) United States
(12) Patent Application Publication
(10) Pub. No.: US 2022/0169582 A1
(43) Pub. Date: Jun. 2, 2022

(54) ORGANIC WASTE DISPOSAL PLANT AND METHOD

(71) Applicant: LIFE BREATH S.R.L., Torino (IT)

(72) Inventor: Alessandro Gazzetta, Torino (IT)

(21) Appl. No.: 17/601,006

(22) PCT Filed: Apr. 1, 2020
(86) PCT No.: PCT/IB2020/053089

§ 371 Date: Oct. 1, 2021

(30) Foreign Application Priority Data
Apr. 3, 2019 (IT) 102019000005016

(51) Int. Cl. C07C 1/12; C25B 1/04; C01B 3/24; C01B 3/50; B01D 39/20; F01N 3/08; B01J 23/755; B01J 23/745; B01J 23/72; B01J 35/08; B01J 35/02; B01J 8/00; B01J 8/06; B01J 19/00

(52) U.S. Cl. CPC C07C 1/12; B82Y 40/00; C01B 3/24; C01B 3/501; B01D 39/2093; F01N 3/0857; B01J 23/755; B01J 23/745; B01J 23/72; B01J 35/08; B01J 35/023; B01J 8/001; B01J 8/006; B01J 8/067; B01J 19/0013; C07C 2523/755; C01P 2006/82; C01B 2203/06; C01B 2203/085; C07C 2521/16; C07C 2523/72; C07C 2523/745; C25B 1/04

(57) ABSTRACT

The treatment plant comprises: a reactor for the sublimation of organic material in order to obtain a syngas; a filtration assembly for filtering the syngas in order to obtain a filtered gas, and a motor-generator assembly for producing electrical energy by means of the combustion of the filtered gas and thereby producing burnt gas; characterized in that said plant also comprises a methanation assembly, comprising: a catalyst that can extract carbon dioxide and nitrogen from the burnt gas; an electrolyzer that can separate water into oxygen and hydrogen by means of electrolysis; and a methanation reactor, which can produce methane by means of the Sabatier reaction using hydrogen and carbon dioxide originating from the electrolyzer and from the catalyst; the catalyst comprising a catalysis layer consisting of stone wool and nickel nanospheres, a plurality of steel microtubes containing copper microfilaments, and a system for controlling the reaction conditions.

The present invention generally relates to a treatment plant for disposing of organic material, and in particular to a plant for disposing of and co-generating electrical and thermal energy.

Treatment plants for disposing of organic material are widely known, which generally comprise: a reactor, adapted to carry out a chemical reaction to obtain a syngas from the organic material; a filtration assembly, adapted to remove a fraction of polluting components from said syngas, in particular “char” (carbonaceous solids) and “tar” (carbonaceous liquids typically containing tarry aromatic hydrocarbons, carbon dioxide and nanoparticulate matter); and a motor-generator assembly, adapted to produce electrical energy by means of the combustion of filtered gas provided by the filtration assembly.

In these known plants, a polluting component is released into the environment in the form of a gas, liquid or solid in any case. Indeed, these known plants do not make it possible to completely close the disposal cycle.

In particular, in known plants, the tar, char and tarry elements can be reduced by up to a maximum of 55%, which causes rapid wear of the components of the motor-generator assembly, which therefore has to undergo frequent maintenance work.

Furthermore, due to the fact that the temperature in the reactor is kept below 800° C., in the known treatment plants it is possible for dioxins, particularly polluting dioxins, to form. Lastly, in the reactors of known plants for treating organic material, accelerator substances are often added in order to reach higher reaction temperatures, which increases the costs and complexity of use of these plants.

The object of the present invention is to provide a treatment plant for the disposal of organic material that does not have the disadvantages of the prior art given above.

This and other objects are achieved according to the present invention by means of a treatment plant as defined in independent claim 1 attached and by means of a treatment method for disposing of organic material as defined in independent claim 8 attached.

Advantageous embodiments of the invention are specified in the dependent claims, the subject matter of which is to be intended to be an integral part of the following description.

In short, the invention is based on the idea of providing a treatment plant for disposing of organic material, comprising:
a reactor, comprising a plurality of electric heating elements adapted to heat a volume inside the reactor to at least 800° C. and to perform a sublimation reaction of said organic material to obtain a syngas,
a filtering assembly in fluid communication with said reactor so as to receive said syngas therefrom, said filtering assembly being adapted to filter the syngas to obtain a filtered gas, and
a motor-generator assembly, comprising a heat engine and an associated generator, adapted to produce electric energy by means of the combustion of the filtered gas provided by the filtering assembly, the heat engine comprising an outlet for discharging a burnt gas produced by the combustion of the filtered gas,
a methanation assembly, in turn comprising
   a catalyst in fluid communication with said outlet of the heat engine, said catalyst being adapted to extract carbon dioxide (CO₂) and nitrogen (N₂) from the burnt gas,
   an electrolyzer, adapted to be supplied with water and to separate said water into oxygen (O₂) and hydrogen (H₂) by means of electrolysis, and
   a methanation reactor that is in fluid communication with the catalyst for receiving carbon dioxide (CO₂) therefrom and is in fluid communication with the electrolyzer for receiving hydrogen (H₂) therefrom, said methanation reactor being adapted to produce methane (CH₄) by means of the Sabatier reaction, characterized in that the catalyst of the methanation assembly comprises
a first catalyst element comprising a catalysis layer of stone wool and nickel nanospheres,
a second catalyst element, which is downstream of the first catalyst element, comprising a plurality of steel micro-tubes containing copper micro-filaments, and
a system for controlling the reaction conditions, which is downstream of the second catalyst element and which is adapted to cause variations in temperature and pressure, according to a predetermined cycle, in the catalyst in order to extract nitrogen (N₂) and carbon dioxide (CO₂) from the burnt gas (GC).

By virtue of a configuration of this type, the treatment plant according to the invention does not have the disadvantages of the prior art. In particular, the plant may convert organic material into syngas up to approximately 100%, therefore producing an amount of process residues that is equal to the quantity of inert material that is initially contained in the organic material supplied to the reactor. As a result, a treatment plant according to the invention does not emit any solid, liquid or gaseous polluting agents into the external environment.

Secondly, by virtue of the presence of a methanation assembly, the disposal plant according to the invention may also provide synthetic methane.

Additional features and advantages of the present invention will become clearer from the following detailed description, which is given purely by way of non-limiting example, with reference to the attached drawings, in which:

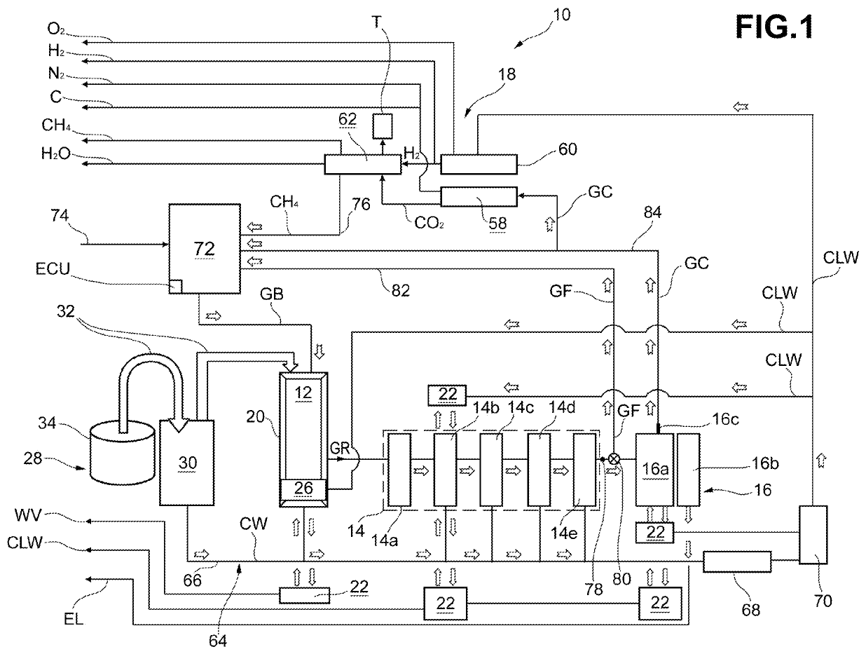

FIG. 1 is a block diagram of a treatment plant according to one embodiment of the invention;

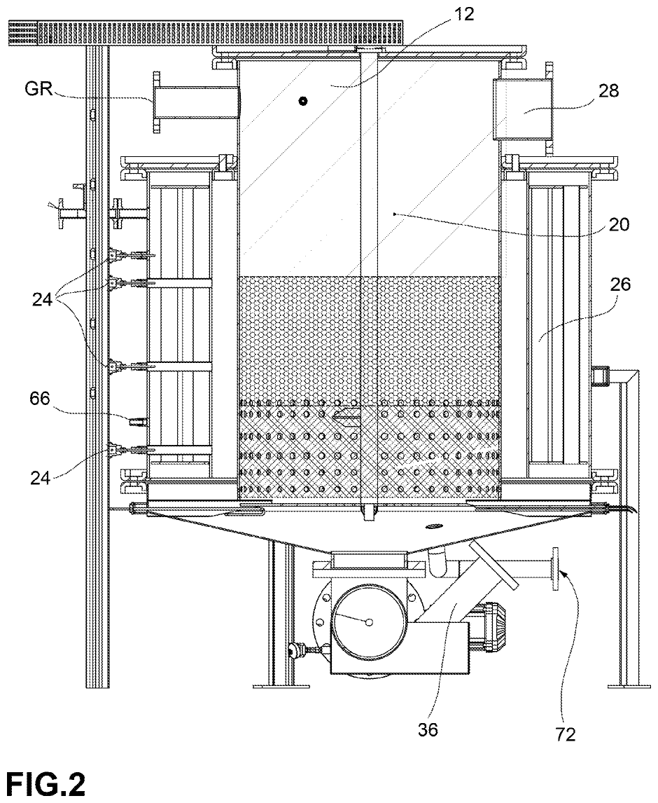

FIG. 2 shows a reactor of a treatment plant according to one embodiment of the invention;

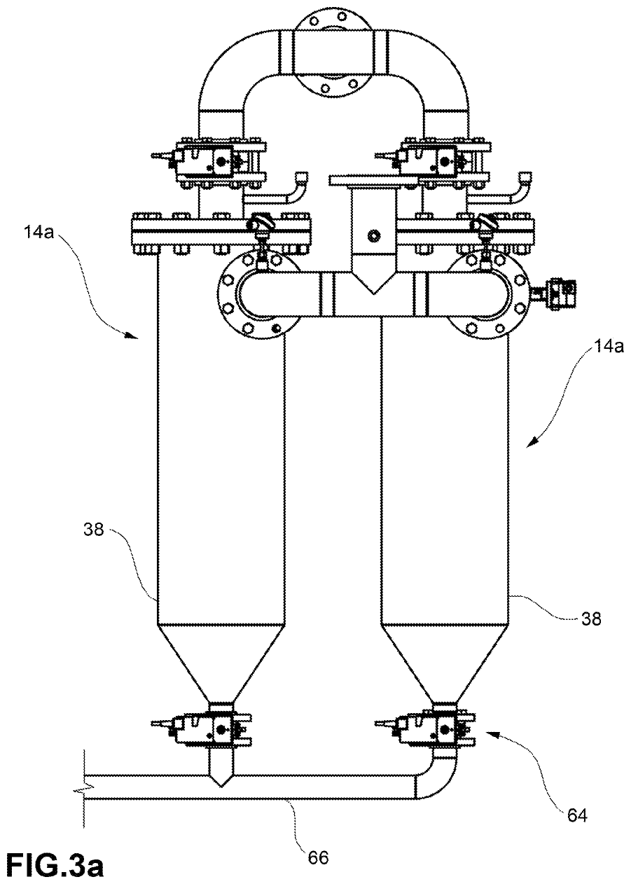

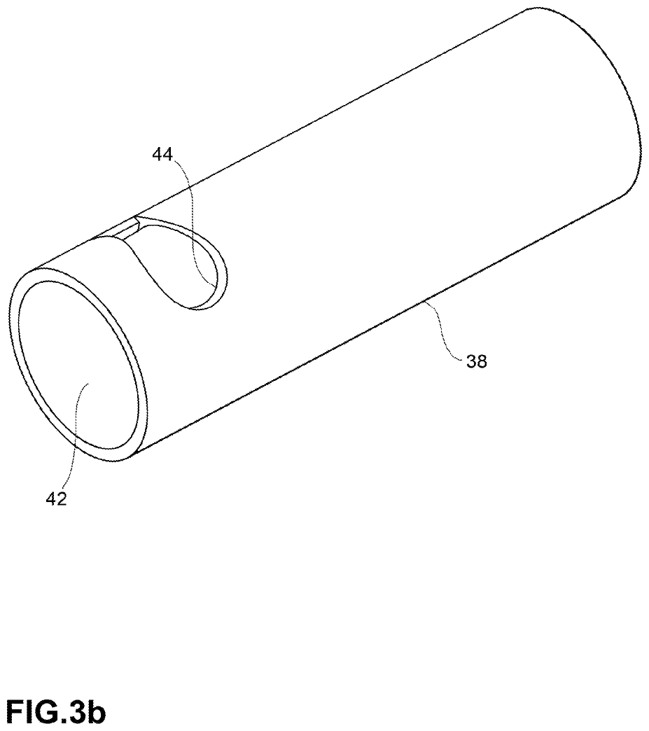

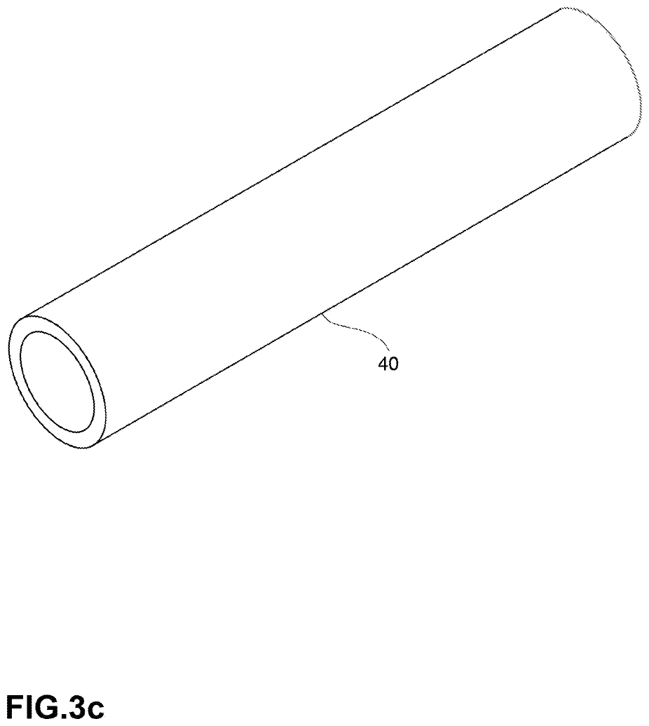

FIGS. 3a, 3b and 3c are a lateral view of two first filtering elements and perspective views of two components of a first filtering element of a filtration assembly of a treatment plant according to one embodiment of the invention, respectively;

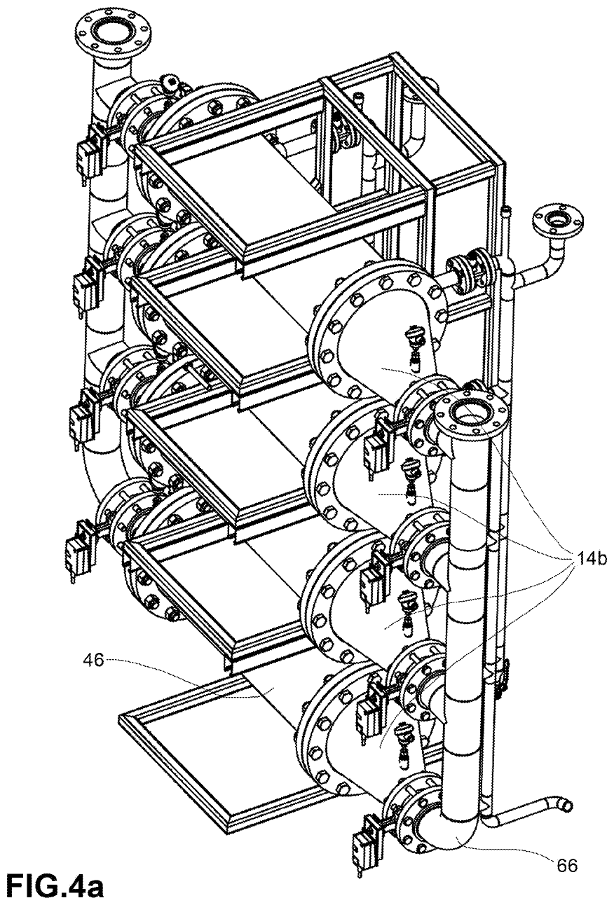

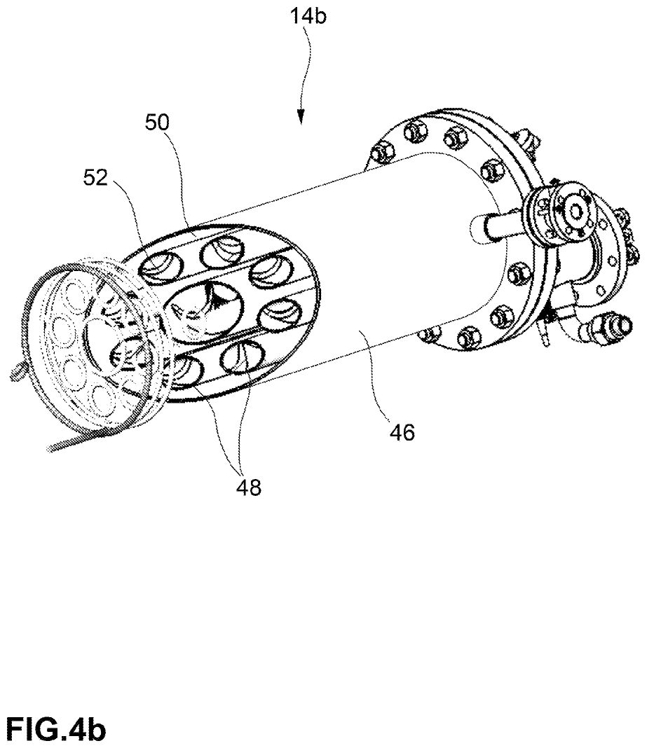

FIGS. 4a and 4b are a perspective view of four second filtering elements and a partially sectional perspective view of a component of the second filtering element of a filtration assembly of a treatment plant according to one embodiment of the invention, respectively; and

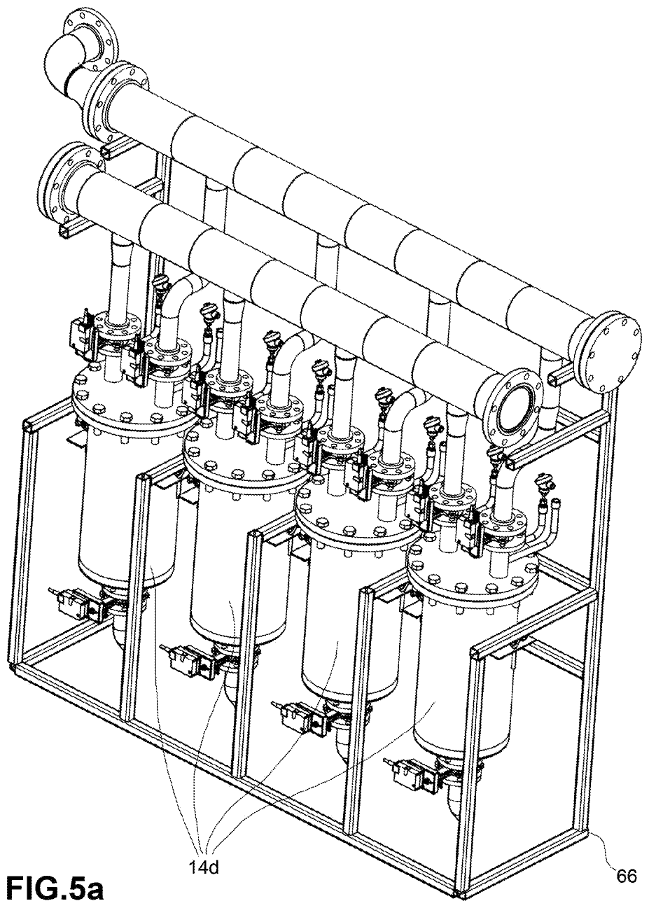

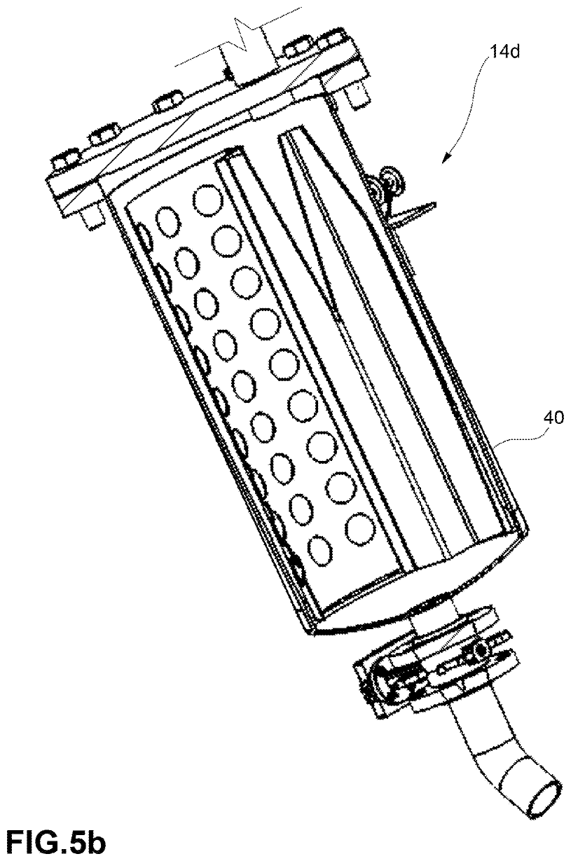

FIGS. 5a and 5b are a perspective view of four fourth filtering elements and a perspective view of a component of the fourth filtering element of a filtration assembly of a treatment plant according to one embodiment of the invention respectively.

Within the context of the present description and the attached claims, the term “organic waste” or “organic material” includes materials predominantly comprising organic compounds, that is, compounds in which one or more carbon atoms are joined to other atoms by means of covalent bonds. Here, materials comprising predominant mass fractions of: wood, pollen, pruning clippings, biomass from cultivation, animal waste, solid urban waste, organic fraction of solid urban waste, disposal waste, refinery sludge, plastics materials, particularly unwanted plastics materials, organic materials, bioplastic materials, microplastic materials, rubber and tires are therefore considered to be organic material, purely by way of non-limiting example.

With reference to the attached drawings, a treatment plant 10 comprises a reactor 12, a filtration assembly 14, a motor-generator assembly 16 and a methanation assembly 18.

In addition, the treatment plant 10 may comprise an electronic control unit ECU adapted to coordinately monitor and control all, or some, of the components of the treatment plant 10. The electronic control unit ECU may be adapted for operating depending on different design parameters, both selected by an operator and predetermined by dedicated software, and, in particular, may be adapted to optimize the operation of the treatment plant 10 on the basis of the amount of organic material used, on the percentage of water that said material contains, and on the desired electric power.

The reactor 12 is adapted to carry out a sublimation reaction of organic material in order to obtain a syngas GR. Within the context of the present invention, “sublimation reaction” means a flameless combustion process of said organic material, carried out in a sub-stoichiometric regime, that is, in the absence of oxygen. Said syngas GR is a gas mixture generally comprising carbon dioxide, carbon monoxide, hydrogen, nitrogen, aqueous vapours, methane and other polluting components in variable fractions depending on the reaction conditions inside the reactor 12, the type of organic material used and the humidity level therein.

The reactor 12 comprises a reaction tank 20, which is typically cylindrical and made of a metal material. In particular, the reaction tank 20 may be made of steel, for example stainless steel AISI 316. Advantageously, in order to tolerate the high temperatures and the specific chemical conditions, at least one inner surface of the reaction tank 20 is made of stainless steel AISI 310.

The reactor 12 also comprises a plurality of electric heating elements (not shown, known per se) that are operatively associated with the reaction tank 20 in order to heat a volume inside the reaction tank 20 to a temperature of 800° C., which is the minimum reaction temperature necessary for preventing the formation of dioxins. The plurality of electrical heating elements are preferably adapted to heat the inside of the reaction tank 20 to a temperature of 1150-1250° C. The plurality of electrical heating elements are preferably arranged so as to heat different overlapping portions of the reaction tank 20 to different temperatures, thereby defining an upper portion, which is heated to a temperature in the range of 500-600° C., an intermediate portion, which is heated to a temperature in the range of 750-800° C., and a lower portion, which is heated to a temperature in the range of 950-1000° C., for example.

A plurality of temperature sensors 24 may advantageously be associated with the reaction tank 20, each of which can measure a temperature inside the reaction tank 20.

Furthermore, a fluid heat exchanger 26 may be associated with the reaction tank 20, which is arranged so as to be in contact with the reaction tank 20 in order to recover excess thermal energy from the reactor 12.

At least one shaker member (not shown, known per se) is advantageously arranged inside the reaction tank 20, which may mix the organic material inside the reaction tank 20 in order to prevent the formation of compact waste material on the bottom of the reaction tank 20, for example a rotary shaker member comprising a plurality of blades.

A loading system 28 comprising an organic material tank 30, which may contain the organic material before it is transported into the reaction tank 20, and a transport mechanism 32 adapted to transport said organic material from the organic material tank 30 into the reaction tank 20, and an iron separator (not shown, known per se) adapted to separate any metal material that is accidentally present in the organic material tank 30 from the organic material are preferably also associated with the reactor 12.

In a manner known per se, the transportation mechanism 32 may comprise a conventional conveyor belt, for example, or a worm transportation mechanism.

A grinding mechanism 34 that can grind the organic material is advantageously associated with the loading system 28.

An inert material removal system 36 is advantageously also associated with the reaction tank 20, adapted to remove inert or non-combustible material from the inside of the reaction tank 20 in order to improve the general efficiency of the sublimation reaction. Said inert material removal system 36 is preferably arranged associated with a bottom wall of the reaction tank 20.

The filtration assembly 14 is arranged in fluid communication with said reaction 12 in order to receive said syngas GR therefrom, and to filter said syngas GR in order to obtain a filtered gas GF. Within the context of the present invention, and in particular of the filtration assembly 14, the term “filter” means the process of removing an unwanted fraction of components of the syngas GR and obtaining filtered gas GF. The unwanted fraction comprises polluting components, such as char and tar mentioned above, possible powders in suspension, ash, tarry components, fine powders, nitric oxides and other components of polluting compounds that are generally produced in a sublimation process at a high temperature.

In a preferable embodiment of the invention, the filtration assembly 14 comprises a first filtering element 14a, a second filtering element 14b arranged downstream of the first filtering element 14a, a third filtering element 14c arranged downstream of the second filtering element 14b, a fourth filtering element 14d arranged downstream of the third filtering element 14c, and a fifth filtering element 14e arranged downstream of the fourth filtering element 14e. Several of each of the filtering elements 14a-14e may be provided in parallel in the treatment plant 10 according to the invention in order to ensure redundancy and, therefore, to keep the treatment plant 10 operational, even in the event of maintenance of, damage to or temporary removal of one of the filtering elements 14a-14e. For example, as shown in FIGS. 3a, 4a and 5a, respectively, a filtration assembly 14 may comprise two first filtering elements 14a, four second filtering elements 14b, and four fourth filtering elements 14d. Furthermore, each of the filtering elements 14a-14e comprises, at least in the structural components thereof that are exposed to the greatest amount of wear, the use of materials that are chemically resistant to exposure to chlorides and sulphides.

The first filtering element 14a comprises a first radially external cylinder 38 and a first radially internal cylinder 40, arranged on the inside of said first radially internal cylinder 38 (preferably in a concentric fashion) so as to define a first passage 42 there between.

In a lateral surface thereof, the first radially external cylinder 38 comprises a mouth 44, adapted to allow syngas GR to pass into the first passage 42. The syngas GR therefore moves in a swirling motion and is pushed towards the radially internal region of the first passage 42.

The first radially internal cylinder 40 is made of a permeable microporous ceramic material. In this way, the syngas GR may pass from the first passage 42 to the inside of the first radially internal cylinder 40. During this filtration process, the syngas GR deposits some of the tarry elements it contains on the outer surface of the first radially internal cylinder 40, that is on a surface of the first radially internal cylinder 40 that faces the first passage 42; in this filtration process, it is said tar that, due to its viscosity, attracts and holds some of the fine powders and the nitric oxides with it, thereby facilitating the deposition thereof on the outer surface of the first radially internal cylinder 40. For example, the TOC (Total Organic Carbon) content of the fluid and that of sulphur oxides may be approximately halved by the first filtering element 14a.

The flow of syngas GR is supplied from the first filtering element 14a to the second filtering element 14b.

The second filtering element 14b comprises a second radially external cylinder 46 and a plurality of second radially internal cylinders 48, which are arranged on the inside of the second radially external cylinder 46 so as to define a second passage 50 there between adapted to convey a liquid. The second filtering element 14b is substantially formed as a conventional shell and tube heat exchanger.

The second passage 50 conducts a liquid for the heat exchange process, for example a condensation liquid. The heat exchange process that takes place inside the second filtering element is adapted to provide a “thermal shock”, that is a strong and sudden decrease in the temperature of the syngas GR so as to prevent the formation of dioxins.

The thermal shock may preferably bring the temperature of the syngas GR that, when it enters the second filtering element 14b is in the range of from 400 to 500° C., to a temperature of between 40 and 75° C. as it leaves the second filtering element 14b. In fact, the second radially internal cylinders 48 conduct the syngas GR that the second filtering element 14b has received from the first filtering element 14a in the interior thereof; the passage of syngas GR inside the second radially internal cylinders 48 takes place at a generally high speed and relatively low pressure. Furthermore, respective turbulators 52 are provided inside each second radially internal cylinder 48. The turbulators 52 are rotary elements, each rotatable about an axis that is concentric with that of each second radially internal cylinder 48, having a metal structure, preferably steel, coated with a microporous ceramic material adapted to allow the deposition of additional pollutant components contained in the reaction gas GR—in particular char, tar and sulphur oxides—on the surface of the microporous ceramic material, and to facilitate said deposition process by means of the rotation on themselves and the consequent generation of turbulence in the flow of the syngas GR.

The flow of syngas GR is supplied from the second filtering element 14b to the third filtering element 14c.

The third filtering element 14c will not be described in detail, since it is made in a substantially similar manner to the first filtering element 14a. However, it differs therefrom in that active carbon, which are adapted to facilitate the additional filtering of the flow of syngas GR, and in particular to facilitate the removal of the nitric oxides, sulphides and ammonia components that are contained inside a third radially internal cylinder that is part of the third filtering element 14c.

The flow of syngas GR is supplied from the third filtering element 14c to the fourth filtering element 14d.

The fourth filtering element 14d will not be described in detail either, since it is made in a substantially similar manner to the third filtering element 14c.

The flow of syngas GR is supplied from the fourth filtering element 14d to the fifth filtering element 14e.

The fifth filtering element 14e will not be described in detail either, since it is made in a substantially similar manner to the third filtering element 14c. However, the fifth filtering element 14e differs therefrom in that it is also provided with a cooling system adapted to cause the syngas GR to cool down and, therefore, any aqueous vapours contained in suspension in said gas to condense. This cooling system may be designed to only be activated when there is a real need, for example, both depending on an intervention by an external operator and depending on the control of dedicated software and an electronic controller, and also depending on the measurements made by a sensor associated therewith.

The microporous ceramic material used in the filtering elements 14a-14e generally has a degree of porosity of between 10 and 40 PPI, preferably between 20 and 35 PPI, and even more preferably of approximately 30 PPI.

Clearly, in a treatment plant 10 according to the invention, the number and arrangement of the filtering elements 14a-14e may be modified according to conventional practice as per methods known to a person skilled in the art.

Pressure transducers and/or temperature sensors (not shown, known per se) may also be associated with each of the filtering elements 14a-14e, being adapted to determine whether reach filtering element 14a-14e is functioning correctly or incorrectly and therefore to allow the necessary maintenance or substitution of the component. Alarm and/or communication systems may be associated with these pressure transducers and/or temperature sensors for informing an operator of the need for maintenance or substitution of the component, for example communication systems that make use of electronic messages, email or SMS.

After having passed through the filtration assembly 14, the syngas GR has a different composition, and is referred to as “filtered gas GF” hereinafter; this filtered gas GF is supplied to a motor-generator assembly 16 arranged downstream of the filtration assembly 14.

In a manner known per se, the motor-generator assembly 16 comprises a heat engine 16a and a generator 16b that is associated with the heat engine, for producing electrical energy EL by means of the combustion of the filtered gas GF that is supplied to the motor-generator assembly 16.

As is known, a burnt gas GC that comprises a fraction of carbon dioxide (CO₂) and potentially fractions of nitric oxides, of unburned filtered gas and other gaseous components will be obtained following the combustion of the filtered gas GF inside the heat engine 16a. As a result, the heat engine 16a also comprises, in a manner known per se, an outlet 16c for discharging said burnt gas GC produced by the combustion of the filtered gas GF.

A methanation assembly 18 is arranged downstream of the moto-generator assembly 16.

The methanation assembly 18 comprises a catalyst 58, an electrolyser 60 and a methanation reactor 62.

The catalyst 58 is in fluid communication with the motor-generator assembly 16 in order to receive burnt gas GC therefrom, and is in particular in fluid communication with the outlet 16c of the engine 16a. The catalyst 58 is adapted to extract carbon dioxide (CO₂) and nitrogen (N₂) from the burnt gas (GC).

In particular, the catalyst 58 comprises a catalysis reactor, inside of which there are a first catalyst element, comprising a catalysis layer formed by stone wool and nickel nanospheres, for example nanospheres having a diameter of 20 nm, and a second catalyst element arranged downstream of the first catalyst element, comprising a plurality of microtubes, preferably steel microtubes containing microfilaments in the interior thereof. The catalyst 58 also comprises a system for controlling the reaction conditions, adapted to bring about changes in temperature and pressure in accordance with a predetermined cycle. Advantageously, the first catalyst element is made by means of the dispersion of nickel powders, in particular containing nickel nanospheres having a diameter of less than 20 nm, and stone wool powders in water; the water is then completely removed from the compound thus obtained. The ratio between the nickel fraction and the stone wool fraction is approximately between 1 to 3 and 1 to 5, and is preferably approximately 1 to 4, respectively. This compound is therefore positioned at the bottom of the catalysis reactor, near to where the burnt gas GC enters the catalysis reactor. The flow of burnt gas GC therefore successively passes through the first catalyst element and the second catalyst element.

The catalyst 58 is therefore provided for supplying separate flows of nitrogen (N₂) and of carbon dioxide (CO₂) downstream.

The electrolyser 60 is adapted to be supplied with water to separate said water into oxygen (O₂) and hydrogen (H₂) by means of a known electrolysis process (and therefore not described here in detail), and is therefore provided for supplying separate flows of oxygen (O₂) and of hydrogen (H₂) downstream.

The carbon dioxide flow leaving the catalyst 58 and the hydrogen flow leaving the electrolyser 60 are supplied to a methanation reactor 62, conveniently upon mixing.

The methanation reactor 62 makes use of the Sabatier reaction or the Sabatier process in order to obtain methane (CH₄) and water (H₂O) in a manner known per se. The methanation reactor 62 operates at a temperature of between 300° C. and 400° C., preferably at a temperature of 280° C., and at a pressure of between 4 and 6 bar, preferably at a pressure of 5 bar. In a preferable embodiment of the treatment plant 10 according to the invention, the methanation reactor 62 reaches the process temperature solely by making use of the thermal energy in the burnt gas GC supplied from the outlet 16c of the heat engine 16a.

A system for storing the methane is preferably associated with the methanation assembly 18 and comprises a compressor and a methane tank (not shown, known per se).

A vent flare T is advantageously associated with the methanation assembly 18, adapted to burn any methane (CH₄) and/or excess filtered gas GF, or gas that causes unexpected overpressure inside the treatment plant 10.

The treatment plant 10 according to the invention preferably also comprises a system for collecting the condensed water 64, which in turn comprises a pipe system 66, a purifier 68 and a conventional water tank 70.

The pipe system 66 is formed in a manner known per se by means of conventional hydraulic pipes that are suitable for transporting water or liquids at a high temperature or pressure, and adapted to collect condensed water CW from the reactor 12, from the filtration assembly 14 and/or from any other component of the treatment plant 10 that produces condensed water or condensation liquid, CW.

The purifier 68 is in fluid communication with the pipe system 66 and is adapted to be supplied with the collected condensed water CW and to purify it in order to obtain purified water CLW.

In a preferable embodiment, the water supplied to the electrolyser 60 contains or consists of purified water CLW supplied from the system for collecting the condensed water 64.

Similarly, in a preferable embodiment, the liquid conducted inside the second passage 50 of the second filtering element 14b contains or consists of purified water CLW that is supplied from the system for collecting the condensed water 64.

In a manner known per se, the system for collecting the condensed water 64 may also comprise a plurality of heat exchangers 22, for example arranged so as to be in contact with the reactor 12 and/or with one or more of the filtering elements 14a-14e and/or with the motor-generator assembly 16, in order to recover the thermal energy of the condensed water CW collected and to obtain aqueous vapours WV.

The treatment plant 10 also comprises a system 72 for supplying oxidizing gas, which is in fluid communication with the reactor 12, and in particular with the reaction tank 20, such that oxidizing gas GB can enter said plant in a turbulent regime. The oxidizing gas GB comprises at least oxygen and nitrogen, and filtered gas GF, burnt gas GC, syngas GR or methane (CH₄) may optionally be added thereto. The system 72 for supplying oxidizing gas may comprise a centrifugal fan, for example, or similar means for generating a turbulent flow of oxidizing gas GB.

In particular, a system 74 for conveying the external air may be associated with the system 72 for supplying oxidizing gas and may supply external air to the system 72 for supplying oxidizing gas.

Similarly, a methane duct 76 may be associated with the system 72 for supplying oxidizing gas, which may place the system 72 for supplying oxidizing gas in fluid communication with the methanation reactor 62 so as to supply methane (CH₄) produced by the methanation reactor 62 to the system 72 for supplying oxidizing gas. In this way, it is also possible to use the methane produced within the same disposal cycle of the organic material, thereby forming a closed cycle.

In a preferable embodiment of the treatment plant 10 according to the invention, a sensor 78 and, downstream thereof, a three-way valve 80, are arranged between the filtration assembly 14 and the motor-generator assembly 16. The sensor 78 is adapted to measure the composition of the filtered gas GF, for example by measuring the mass fraction of unwanted components therein, such as the char, tar or other components. The three-way valve 80 is adapted to receive filtered gas GF that is leaving the filtration assembly 14 and to regulate a flow supplied to the reactor 12 and/or to the motor-generator assembly 16 based on the measurements made by said sensor 78. For example, when the sensor 54 determines that the filtered gas GF does not have a suitable composition for being supplied to the motor-generator assembly 16, the three-way valve 80 may be set up so as to close the supply to the motor-generator assembly 16 and open a return duct 82 that permits the supply of filtered gas GF to the reactor 12. Alternatively, the return duct 82 may be provided to supply filtered gas GF to the system 72 for supplying oxidizing gas, for example for possible mixture with external air or methane and/or for obtaining a turbulent regime of the flow before it enters the reaction tank 20.

Furthermore, a recycling duct 84 may be associated with the system 72 for supplying oxidizing air, adapted to place the outlet 16c of the heat engine 16a in fluid communication with the system 72 for supplying oxidizing air so as to supply said system with burnt gas GC produced by the heat engine 16a.

The system 72 for supplying oxidizing air may balance the different components of the oxidizing gas GB. For example, it is possible to introduce an oxidizing gas GB into the reactor 12 that is composed of 100% external air; or 60% external air, 10% syngas GR, 10% methane and 20% burnt gas GC; or 40% external air, 10% syngas GR, 25% methane and 25% burnt gas GC.

A treatment method for disposing of organic material is also part of the invention. As will be clearly deducible from the following description of the method, said treatment method is implementable by means of a treatment plant 10 as described previously. In virtue of this, the treatment method will be described hereinafter with reference to the treatment plant 10 according to the invention and to the components thereof.

In particular, the treatment method according to the invention comprises the steps of:

a) inserting organic material into a reactor 12, and heating a volume inside the reactor 12 by means of the plurality of electric heating elements to at least 800° C. to carry out a sublimation reaction of said organic material and to obtain syngas GR;

b) filtering said syngas GR to obtain filtered gas GF by means of the filtering assembly 14;

c) producing electrical energy by means of the combustion of said filtered gas GF by the heat engine 16a associated with the generator 16b;

d) extracting carbon dioxide (CO₂) and nitrogen (N₂) from the burnt gas GC by means of the catalyst 58;

e) carrying out a process of electrolyzing water by means of the electrolyser 60 to obtain hydrogen (H₂);

f) supplying the carbon dioxide (CO₂) extracted by the catalyst 58 and the hydrogen (H₂) obtained by means of the electrolyser 60 to the methanation reactor 62 in order to produce methane (CH₄) by means of the Sabatier reaction.

In particular, with reference to step a), the plurality of electric heating elements are supplied with electric power and therefore produce heat that is transmitted to the reaction tank 20. Heating is continued as long as it does not reach the trigger point of the sublimation reaction for producing the syngas GR, and, advantageously, until the temperature inside the reaction tank, in particular at the bottom thereof, has reached 1000° C. Upon reaching these conditions, the process stabilizes in a self-maintenance state and can interrupt the supply of electric power to the plurality of electric heating elements.

The treatment method according to the invention may also comprise the steps of:

g) introducing oxidizing gas GB in a turbulent regime into the reactor 12 by means of the system 72 for supplying oxidizing gas; and

h) supplying the system 72 for supplying oxidizing gas with external air by means of the system 74 for conveying external air; and/or

i) supplying the system 72 for supplying oxidizing gas with methane (CH₄) produced by the methanation reactor 62 by means of the methane duct 76; and/or

l) supplying the system 72 for supplying oxidizing gas with filtered gas GF filtered by the filtering assembly 14 by means of the return duct 82; and/or

m) supplying the system 72 for supplying oxidizing gas with burnt gas GC that is discharged by the heat engine 16a by means of the recycling conduit 84.

In particular, with reference to step g), the introduction of oxidizing gas GB into the reaction tank 20 advantageously provides a sub-stoichiometric ratio, that is the introduction of an oxidizing gas GB in the absence of oxygen. Furthermore, by virtue of the generation of suitable turbulence, it is possible to reach a stable temperature inside the reaction tank 20 of approximately 1250° C. by adjusting the speed of the oxidizing gas GB to equal the flow introduced.

Furthermore, the treatment method may also comprise the steps of:

n) collecting condensed water CW from the reactor 12 and/or from the filtering assembly 14; and

o) purifying the collected condensed water CW by means of a purifier 68 in order to obtain purified water CLW; and

p) supplying said purified water CLW to a water tank 70.

In one embodiment of the invention, the treatment method and the operation of the components of the treatment plant 10 that carries out the treatment method are controlled, at least in part, by the electronic control unit ECU, optionally with the aid of timers, sensors and/or devices for measuring the control parameters.

As is clear from the above description, a treatment plant according to the invention guarantees a range of advantages over the prior art.

First, by virtue of the presence of the methanation assembly, a treatment plant according to the invention allows for the production of synthesis methane.

Furthermore, by virtue of the arrangement of the components of the plant, and in particular of the system for collecting the condensed water and of the system for supplying oxidizing gas, the plant and the treatment method make it possible to minimize the waste of a process for treating organic material. In particular, only water, nitrogen, carbon, oxygen, methane, hydrogen, thermal energy and electrical energy escape from a treatment plant according to the invention, all in usable form. In particular, it has been observed that, in one use cycle of the treatment plant according to the invention, an amount of ash is produced in proportions of less than 0.5 kg per 100 kg of dry organic material within a variability range of approximately 7%, depending on the temperature, humidity and bulk density of the combustible organic material used in the treatment method and therefore introduced into the reactor.

Furthermore, in the plant and the treatment method according to the invention, it is not necessary to use reaction accelerators, which has clear savings in terms of costs and ease of management of the process.

The treatment plant according to the invention can be suitably dimensioned and customized, depending on the scale required, based on the user's needs. Lastly, all the materials used in the filtration assembly are suitably recyclable. Of course, without prejudice to the aim of the invention, the embodiments and details may be largely varied with respect to what has been described and illustrated purely by way of non-limiting example, without thereby departing from the scope of the invention, as defined in the attached claims.

1. A waste processing plant for disposal of organic material, comprising:
a reactor, comprising a plurality of electric heating elements adapted to heat at least a volume inside the reactor to at least 800° C. and to perform a sublimation reaction of said organic material to obtain a synthetic gas,
a filtering assembly in fluid communication with said reactor for receiving from the latter said synthetic gas, said filtering assembly being adapted to filter the synthetic gas to obtain a filtered gas, and
a motor-generator unit, comprising a heat engine and an associated generator, adapted to produce electric energy by means of the combustion of the filtered gas provided by the filtering assembly, the heat engine comprising an outlet for discharge of a burnt gas produced by the combustion of the filtered gas, and
a methanation assembly, comprising
a catalyst in fluid communication with said outlet of the heat engine, said catalyst being adapted to extract carbon dioxide and nitrogen from the burnt gas,
an electrolyser, adapted to be supplied with water and to separate said water into oxygen and hydrogen by means of a process of electrolysis, and
a methanation reactor, in fluid communication with the catalyst for receiving carbon dioxide from it, and in fluid communication with the electrolyser for receiving hydrogen from the latter, said methanation reactor being adapted to produce methane by means of the Sabatier reaction;
characterized in that the catalyst of the methanation assembly comprises:
a first catalytic element comprising a catalyst layer of stone wool and nickel nanospheres,
a second catalytic element, downstream the first catalytic element, comprising a plurality of steel micro-tubes, containing copper micro-filaments, and
a reaction conditions control system, downstream the second catalytic element, adapted to cause variations of temperature and pressure, according to a predetermined cycle, in the catalyst, in order to extract nitrogen and carbon dioxide from the burnt gas.

2. A waste processing plant according to claim 1, wherein the filtering assembly comprises
a first filtering element, comprising a first radially outer cylinder and a first radially inner cylinder arranged concentrically so as to define a first passage there between, said first radially inner cylinder being permeable to a gas circulating in said first passage and being made of microporous ceramic material,
a second filtering element, arranged down-stream of the first filtering element, comprising a second radially outer cylinder and a plurality of second radially inner cylinders, arranged inside the second radially outer cylinder, so as to define a second passage there between adapted to convey a liquid, said second radially inner cylinders being arranged to convey the synthetic gas, each one of said second radially inner cylinders hosting a turbulator internally, each turbulator being adapted to generate a turbulence of the flow there-in, and each turbulator having a coating in microporous ceramic material,
a third filtering element, downstream the second filtering element, comprising a third radially outer cylinder and a third radially inner cylinder arranged concentrically so as to define a third passage there between, said third radially inner cylinder being permeable to a gas circulating in said third passage and being made of microporous ceramic material, the third radially inner cylinder further accommodating active carbons,
a fourth filtering element, downstream the third filtering element, comprising a fourth radially outer cylinder and a fourth radially inner cylinder arranged concentrically so as to define a fourth passage there between, said fourth radially inner cylinder being permeable to a gas circulating in said fourth passage and being made of microporous ceramic material, the fourth radially inner cylinder further accommodating active carbons, and
a fifth filtering element, downstream the fourth filtering element, comprising a fifth radially outer cylinder and a fifth radially inner cylinder arranged concentrically so as to define a fifth passage there between, said fifth radially inner cylinder being permeable to a gas circulating in said fifth passage and being made of microporous ceramic material, the fifth radially inner cylinder further accommodating active carbons, and the fifth filtering element being provided with a cooling system to eliminate possible condensate.

3. A waste processing plant according to claim 2, further comprising a condensate water collection system comprising
a piping system arranged to collect condensate water from the reactor and/or from the filtering assembly,
a purifier arranged to receive the collected condensate water and to purify it obtaining purified water, and
a water reservoir adapted to store the purified water.

4. A waste processing plant according to claim 3, characterized in that said water supplied to the electrolyser contains purified water supplied by the condensate water collection system.

5. A waste processing plant according to claim 3, characterized in that said liquid conveyed into the second passage of the second filtering element contains condensate water supplied by the condensate water collection system.

6. A waste processing plant according to claim 1, further comprising
an oxidising gas supply system, in fluid communication with the reactor to supply oxidising gas in a turbulent regimen into the reactor,
an external air conveying system arranged to supply the oxidising gas supply system with external air, and
a methane conduit adapted to put the oxidising gas supply system in fluid communication with the methanation reactor, in such a way to supply the oxidising gas supply system with methane produced by the methanation reactor, and/or
a return conduit adapted to put the filtering assembly in fluid communication with the oxidising gas supply system, in such a way as to supply the oxidising gas supply system with filtered gas filtered by the filtering assembly, and/or
a recirculation conduit adapted to put the outlet of the heat engine in fluid communication with the oxidising gas supply system, in such a way as to supply the oxidising gas supply system with burnt gas produced by the heat engine.

7. A waste processing plant according to claim 1, further comprising
a sensor adapted to measure the composition of the filtered gas downstream the filtering assembly, and
a three-way valve arranged downstream the filtering assembly, adapted to receive filtered gas flowing out of the filtering assembly and to adjust a flow rate supplied to the reactor and/or to the motor-generator unit, as a function of the measurement of said sensor.

8. A processing method for disposal of organic material, comprising the steps of:
a) inserting organic material in a reactor, and heating a volume inside the reactor by means of a plurality of electric heating elements to at least 800° C. to perform a sublimation reaction of said organic material and obtain a synthetic gas;
b) filtering said synthetic gas to obtain a filtered gas by means of a filtering assembly in fluid communication with said reactor for receiving from the latter said synthetic gas;
c) producing electric energy by means of the combustion of said filtered gas, obtained in step b), performed by a heat engine associated to a generator, the heat engine comprising an outlet for discharge of a burnt gas produced by the combustion of the filtered gas;
d) extracting carbon dioxide and nitrogen from the burnt gas by means of a catalyst in fluid communication with said out-let of the heat engine;
e) performing a process of electrolysis of water by means of an electrolyser to obtain hydrogen;
f) supplying the carbon dioxide extracted by the catalyst and the hydrogen obtained by means of the electrolyser to a methanation reactor to produce methane by means of the Sabatier reaction.

9. A processing method according to claim 8, further comprising the steps of:
g) inserting oxidising gas in turbulent regimen in the reactor, by means of an oxidising gas supply system, in fluid communication with the reactor, and
h) supplying the oxidising gas supply system with external air by means of an external air conveying system, and/or
i) supplying the oxidising gas supply system with methane produced by the methanation reactor by means of a methane conduit, and/or
l) supplying the oxidising gas supply system with filtered gas filtered by the filtering assembly by means of a return conduit, and/or
m) supplying the oxidising gas supply system with burnt gas discharged by the heat engine, by means of a recirculation conduit.

10. A processing method according to claim 8, further comprising the steps of:
n) collecting condensate water from the reactor and/or from the filtering assembly by means of a piping system, and
o) purifying the collected condensate water by means of a purifier to obtain purified water, and
p) supplying said purified water to a water reservoir arranged to contain the purified water.

11. A processing method according to claim 8, carried out by means of a waste processing plant.
